(19) United States
(12) Patent Application Publication
(10) Pub. No.: US 2020/0330370 A1
(43) Pub. Date: Oct. 22, 2020

(54) METHOD FOR TOPICAL SOLVENT-FREE AROMATIC DRUG DELIVERY SYSTEM

(71) Applicant: Reza Fatahi, Encino, CA (US)

(72) Inventor: Reza Fatahi, Encino, CA (US)

(21) Appl. No.: 16/389,935

(22) Filed: Apr. 20, 2019

(51) Int. Cl. A61K 9/00; C11B 9/00; A61K 47/10; A61K 47/26; A61K 47/20; A61K 47/24

(52) U.S. Cl. CPC A61K 9/0014; C11B 9/00; A61K 47/24; A61K 47/26; A61K 47/20; A61K 47/10

(57) ABSTRACT

We report a novel method to replace solvents during the manufacturing of liquid topical oil and water emulsions for therapeutic purposes. The product of this invention is the lowest toxicity strategy to replace solvents during the manufacturing of sprayable liquid oil and water emulsified topicals.

This application is a continuation-in-part of application Ser. No. 16/270,606, filed Feb. 8, 2019 and application Ser. No. 16/294,852, filed Mar. 6, 2019.

### FIELD OF THE INVENTION

The present invention relates to solvent-free preparations of deodorising and fragrance releasing emulsions as a vehicle for the topical application of therapeutic substances. The result of this invention are liquid oil and water products unique in appearance and behavior since they allow a natural separation of oil from water, while also allowing full and partial emulsification following mechanical agitation.

### BACKGROUND OF THE INVENTION

Solvents are short carbon chain molecules that are highly volatile, and flammable under standard conditions. Solvents are commonly used in mass consumer goods to mix or dissolve oil- and water-soluble substances.

Solvents have become a public health concern due to their overabundance in mass consumer goods. Long-term exposure to solvents can cause damage to cognitive, reproductive, renal, hepatic, respiratory, immunological, endocrine, and cardiac functions in mammals.

Solvents irritate live tissues, and their presence in fragrances is not tolerated by mammals with sensitive smell. For this reason colognes and perfumes are often disallowed in the workplace, and settings such as hospitals and clinics.

Consumers are limited in their choice of solvent-free preparations, especially in the art of fragrances, personal care products, and other topical preparations. The state of the art for effective solvent-free, and safe, products are pure oil and pure aromatic water preparations. However, aromatic waters do not have a persistent fragrance, and are limited in their solubility of compounds. Furthermore, oils cannot be sprayed, leave an oily residue that can trap dirt and cause grime, and are also limited in their ability to solubilize compounds.

It has recently been uncovered that solvents damage the therapeutic potentials of active ingredients. The present invention serves to retain the therapeutic potential of active ingredients by substituting solvents with the lowest toxicity strategy defined herein.

### SUMMARY OF INVENTION

The present invention determines the lowest toxicity substitute for solvents during the manufacturing of oil and water emulsions for therapeutic topical spray products. A system is defined to identify the emulsification properties of active ingredients. The emulsifier blend described in the prior art of this application is modulated by the presence of active ingredients with emulsification properties.

Glutathione and silybin are used to illustrate the methodology of this invention. Glutathione is an antioxidant found in plants, animals, fungi, and bacteria. Silybin is the major active constituent of silymarin, a standardized extract of the milk thistle seeds, which is a therapeutic substance for the treatment of liver and gallbladder disorders.

Glutathione prevents damage to important cellular components and processes caused by oxidation. The chemical structure of glutathione contains nine carbon atoms that are bridged by polar pnictogens and chalcogens. Although this chemical has polarity (electrical imbalance), the polarity is biased towards one end, thus, mimicking the esterification of soaps. Furthermore, the arrangement of carbon atoms in the chemical structure of glutathione is balanced. There are few branches, and an absence of toxic qualities including, and not limited to the absence of highly polar halogens, short carbon chain arrangement, and high number of branches.

Silybin has a chemical structure that contains multiple aromatic carbon rings, and multiple pseudo carbon rings bridged by chalcogens. The present invention allows bridged carbon rings with non-halogens to be considered as a continuous carbon ring structure for consideration of emulsification properties based on the prior art of this application. With this consideration, silybin has few or no branching as it relates to the present invention.

Both glutathione and sylibin express soap-like characteristics that support emulsification-like properties that may be considered during the preparation of the emulsifier blends for topical formulations. This method of manufacturing applies to other chemicals during formulation for specific use cases. Therefore, the present invention does not limit the modulation of emulsifier blends to glutathione or sylibin.

### BRIEF DESCRIPTION OF THE DRAWINGS

FIG. 1: The chemical structure of glutathione. The red circles define the so-called bridge, here as pnictogen bridges, that under the present invention are reduced to a continuation of the long carbon chain. There are two faces to the molecule consisting of a more polar and less polar surface, thus, providing the basis for emulsification properties in native and/or esterified forms. Furthermore, there is an overall, yet imperfect, balance to the molecule whereby reduced volatility may be implied.

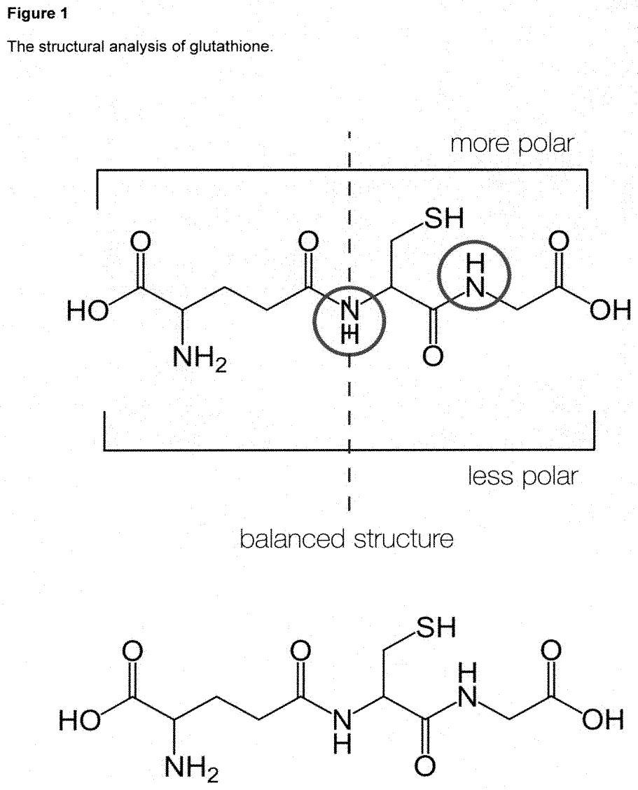

FIG. 2: The chemical structure of silybin. The red circles define the so-called bridge, here as pnictogen bridges, that under the present invention are reduced to a continuation of the long carbon chain. The green circles define chiral centers with less than three bonds to carbon rings that are reduced to a continuation of the long carbon chain. Sylibin is imperfectly balanced structurally and electrically, thus, providing a basis for natural and/or esterified emulsification properties, and low volatility behavior.

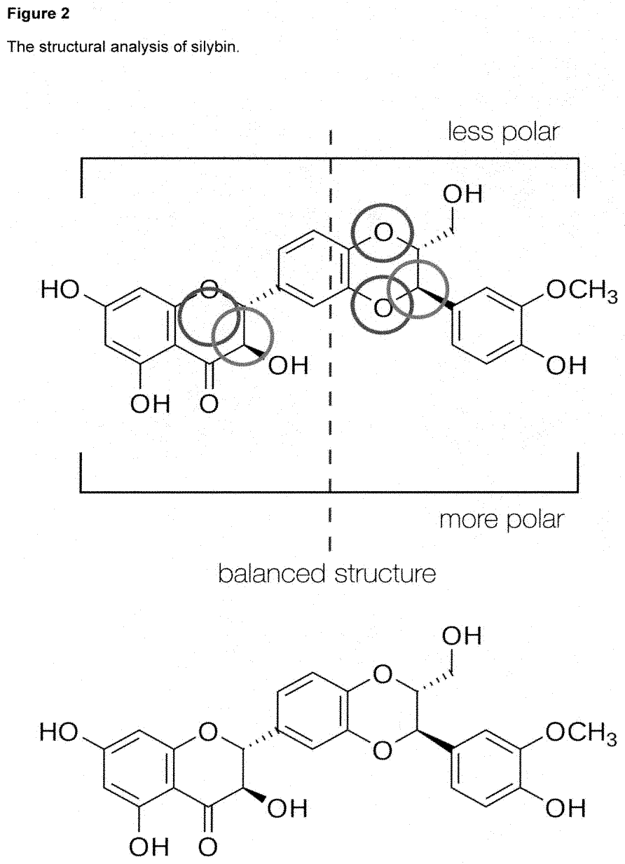

FIG. 3: The unique appearance of the products of this invention. (A) The product in its settled state. Note the separation of oil and water layers. The degree of separation may vary. (B) The product in its emulsified state. Note the homogenization of oil and water. The degree of homogenization may vary.

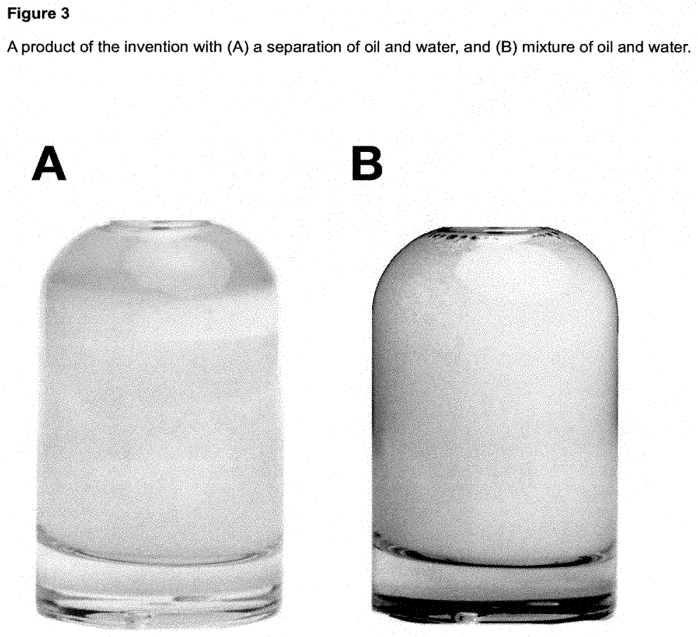

### DETAILED DESCRIPTION OF THE INVENTION

The rules for establishing emulsifier candidacy under the present invention relies on a formulation for therapeutic purposes that contains one or more therapeutic active ingredients. A determination of emulsifier-like properties essentially consists of:
1) a carbon based molecule that is six or more carbon atoms in length at its longest edge,
2) molecules that contain a more polar facet and a less polar facet along a two dimensional plane, and
3) molecules that are generally structurally balanced, even if not perfectly balanced.

The emulsifier blend of the prior art consists of esterified fatty-acid or like molecules that essentially consist of a carbon chain of six or more carbon atoms, and up to four branches consisting of two or more carbon atoms in length per branch. Active ingredients may be used to augment or modulate the emulsifier blend by reducing the amount or replacing esterified fatty-acid or like molecules.

The present invention covers the emulsification properties of esterified and non-esterified active ingredients. In order for non-esterified active ingredients to be considered, they must have a more polar surface or facet relative to a less polar surface or facet (along a two-dimensional plane).

Furthermore, the present invention also applies novel rules for considering the continuity of carbon chains, and extends the following rules:
1) carbon atoms separated by a single chalcogen, pnictogen, or like atom are considered to be a continuation of the carbon chain, and
2) carbon rings that are bound by one or more chalcogens, pnictogens, or like atoms are considered to be a contiguous carbon ring.

The present invention considers the bridging of two carbon atoms by chalogens, pnictogens, and like atoms to count as a continuation of the carbon chain. This consideration also applies to carbon rings (cyclical carbon arrangements) wherein bridging may occur between two carbon atoms.

Furthermore, carbon rings are not considered as separate branched structures unless three or more carbon rings are bound to the branch origin point. Neighboring carbon rings are also considered to be a continuation of a single carbon chain.

A developing trend in the literature is about the damages caused by solvents to classes of chemicals including anti-inflammatory, pesticidal, and nutritional substances. The prior art has not addressed a viable alternative to solvents for topical oil and water sprayable emulsions. Furthermore, the prior art has not addressed a substitute for solvents as a method to retain the therapeutic potential of active ingredients.

The products of the present invention are liquid and sprayable oil and water emulsions as a vehicle for the topical application of therapeutic active ingredients. This invention eliminates the damages caused by solvents to the active ingredients of oil and water emulsions.

1-24. (canceled)

25. A novel non-translucent aromatic solution composed of water and oil mixed with soap and soap-like materials that can be sprayed through conventional means, and that can contain various oil-soluble and water-soluble substances and materials for beautification and therapeutic effects.

26. A non-translucent fragrance solution according to claim 25 that results from mixing oil and water with esterified medium- and long-chain hydrocarbons, and soap-like materials including detergents, emulsifiers, and surfactants such as polyalkylene glycols, polysorbates, sulfonates, lecithin, and others.

27. A fragrance product made with soap and soap-like materials according to claim 26 that can vary in their compositions, combinations, and concentrations.

28. A fragrance product according to claim 27 whose homogenization may be persistent or not persistent dependent on the type and concentration of soap and soap-like materials used to mix oil and water, which results in the separation of water and oil to one or more layers.

29. A method according to claims 26-28 whose mixing of oil and water result in a single layer non-translucent product that is milky in appearance, and whose function and benefits are derived by its novel homogenized non-translucent appearance.

30. A method according to claims 26-28 whose mixing of oil and water result in a low-viscous solution that does not cause surfaces to become oily.

31. A method according to claims 26-28 whose mixing of random essential oils and water generically results in a lemon scent.

32. A non-translucent fragrance product according to claim 31 that can vary in scent and color based on the essential oil composition, synthetic aromatic oil composition, and water composition (i.e. aromatic and colored waters).

33. A non-translucent fragrance product according to claim 32 whose solubility properties can be altered with the addition of acids, bases, metals, and other materials.

34. A non-translucent fragrance product according to claim 33 that can vary from complete separation of layers to incomplete separation of layers.

35. A non-translucent fragrance product according to claim 34 that often requires mixing prior to use, and where mixing can be accomplished with mild to moderate centrifugal force.

36. A topical drug delivery product that maintains the integrity and therapeutic potential of its components against harms caused by organic solvents such as alcohol.

37. A topical drug delivery product according to claim 36 that can partially or completely replace organic solvents such as alcohol with soap and soap-like materials.

38. A topical drug delivery product according to claim 37 that eliminates the irritation, risk, and damages caused by contacting and inhaling solvents to the skin and live tissues irrespective of age, gender, and species.

39. A topical drug delivery product according to claim 38 that eliminates the damages caused by solvents to the antiseptic potential of essential oils.

40. A topical drug delivery product according to claim 39 that preserves the natural antimicrobial, antifungal, antiparasitic, and antiviral properties of essential oils, and that can be used for cleansing and elimination of odor and disease causing bacteria, fungi, parasites, and viruses.

41. A topical drug delivery product that is multipurpose and non-irritating on inhalation or contact to skin and live tissues, irrespective of age, gender, and species of animal, including humans, and safe to use for therapeutic topical delivery and cleansing to sensitive and insensitive tissues.

42. A topical drug delivery product according to claims 25-42 that can include additional materials such as nanomaterials, nutrients, drugs, probiotics, and other components that will not be damaged by solvents to deliver enhanced therapeutic effects.

43. A topical drug delivery product according to claims 25-42 that is not restricted in its ability to topically deliver existing and novel therapeutic materials, including FDA approved monographs drugs.

44. A novel drug delivery system according to claim 43 that is eligible for new FDA and drug regulatory approvals for therapy.

45. A novel drug delivery system according to claims 43 and 44 that is not restricted for topical therapeutic use by tissue type, age, gender, and species of animals, including humans.

46. A novel drug delivery system according to claims 43-45 that is not restricted by prescription or over-the-counter status availability.
